(19) United States
(12) Patent Application Publication
(10) Pub. No.: US 2023/0405593 A1
(43) Pub. Date: Dec. 21, 2023

(54) MICROFLUIDIC DEVICE AND METHOD FOR ANALYSIS OF A PARTICULATE SAMPLE

(71) Applicant: UNIVERSITY OF SOUTH AUSTRALIA, Adelaide (AU)

(72) Inventors: Craig Priest, Athelstone (AU); Aliaa Shallan, Cairo (EG); Michael Breadmore, Tasmania (AU); Moein Navvab Kashani, Glenunga (AU)

(21) Appl. No.: 18/248,175

(22) PCT Filed: Oct. 8, 2021
(86) PCT No.: PCT/AU2021/051178

§ 371 Date: Apr. 6, 2023

(30) Foreign Application Priority Data
Oct. 8, 2020 (AU) 2020903645

(51) Int. Cl. B01L 3/00; G01N 33/24

(52) U.S. Cl. CPC B01L 3/502753; B01L 3/502707; B01L 3/502761; G01N 33/24; B01L 2200/0668; B01L 2300/04; B01L 2300/0874

(57) ABSTRACT

The present invention relates generally to devices able to manipulate, process, treat, sort, measure and/or analyse samples at a micro level, commonly referred to as microfluidic devices. In particular, the present invention relates to a microfluidic device that can be used for the analysis of particulate samples, such as by the leaching at a micro level of a crushed rock particulate sample from a mineral ore body and the subsequent analysis of the leachate. The present invention also relates to a method for the use of a microfluidic device for the analysis of a particulate sample.

### FIELD OF THE INVENTION

The present invention relates generally to devices able to manipulate, process, treat, sort, measure and/or analyse samples at a micro level, commonly referred to as microfluidic devices. In particular, the present invention relates to a microfluidic device that can be used for the analysis of particulate samples, such as by the leaching at a micro level of a crushed rock particulate sample from a mineral ore body and the subsequent analysis of the leachate. The present invention also relates to a method for the use of a microfluidic device for the analysis of a particulate sample.

### BACKGROUND OF THE INVENTION

A reference to “microfluidics” is typically a reference to the use of physical structures with fluid control features having at least one dimension that is at the sub-millimetre level. If all or a majority of the dimensions of a physical structure are at the millimetre level, the structure would generally be referred to with the prefix milli-, while below this the physical structure would generally be referred to with the prefix nano-. Hence, throughout this specification reference will be made to microchannels, microwalls and micropillars, for example, meaning generally that the channels, walls and pillars are sub-millimetre in dimension. Typically, such channels and pillars will actually be sized less than about 500 micrometres, or less than about 100 micrometres, but certainly greater than 1 micrometre.

Microfluidics is a multidisciplinary field at the intersection of engineering, physics, chemistry, biochemistry, nanotechnology, and biotechnology, with practical applications in the design of systems in which low volumes of fluids, normally liquids, are processed to achieve multiplexing, automation, and high-throughput screening. Microfluidics emerged in the beginning of the 1980s and has been used in areas as diverse as DNA chips, lab-on-a-chip technology, micro-propulsion and micro-thermal technologies.

Conventionally, devices that manipulate liquids in the microscale offer benefits to be used as miniaturized laboratories, such as low energy consumption, shorter chemical reaction time, small sample and biological reagents consumption, low cost, high compactness, high integration and the possibility of multiple tests per device. Also, microfluidic-based devices may facilitate remote and touch-less manipulation of single cells, micro-organisms or micro-particles.

Typically in microfluidic systems, liquids are transported, mixed, separated or otherwise processed. The various applications of such systems rely on passive liquid control using capillary forces, in the form of capillary flow modifying elements, akin to flow resistors and flow accelerators. In some applications, external actuation means are additionally used for a directed transport of the liquid. Examples are rotary drives applying centrifugal forces for liquid transport on passive chips. Alternatively, there can be manipulation of the working liquid by active components such as micropumps or microvalves.

Processes normally carried out in a laboratory can thus, with microfluidics, be miniaturised on a single device in order to enhance efficiency and mobility, as well as to reduce sample and reagent volumes.

Acid mine drainage (AMD) is one of the most significant environmental pollution problems associated with the mining industry. Case-specific testing is widely applied and established in the mining and consulting businesses for AMD prediction, and any improvements in the efficiency of this testing, while reducing the environmental impact of AMD, are thus of societal importance.

AMD forms when sulphide minerals are exposed to oxidizing conditions e.g. water and oxygen. This occurs naturally where sulphide minerals exist in water-saturated zones, but the process can be considerably accelerated by the production of broken waste rock and tailings through mining operations. Sulphide-bearing mining waste can produce large volumes of contaminated effluents with elevated acidity and often contains toxic amounts of dissolved heavy metals (such as Fe, Cu, Mn, Zn and Pb). Due to its severe environmental impact on soil, water resources. and aquatic environments, AMD has become a significant environmental issue facing the mining industry.

The prediction and control of AMD therefore plays a significant role in most strategies for controlling pollution from mining operations. Although the fundamental chemistry of AMD formation has been extensively examined, the resulting profiles of waste rock and tailings are highly dependent on several factors, including geological setting, mineralogy, presence of microorganisms, and other environmental variables such as temperature, oxygen and water. These factors are highly variable for any given mine waste, and therefore, long-term AMD management requires effective and efficient investigation of AMD and a better understanding of leach behaviour of sulphide minerals under actual field conditions at mining sites.

Current practices for an AMD assessment of sulphide-bearing mining wastes primarily involves long-term acid generation/alkalinity definitions under batch, column and drum leach conditions, in order to evaluate the acid generation, sulphate release and metal release rates based on specific sulphide reaction pathways. However, these tests are time-consuming (up to months or years) and are typically plagued with an insufficient spatiotemporal control associated with the large volumes of the reactors normally used. Moreover, accurate control of the essential physio-chemical variables of such large-scale experiments, such as temperature, are unrealistic over the longer term, and screening measurements that cover the necessary physical or chemical properties require a relatively large quantity of samples and reagents.

Additionally, natural formations (such as minerals) are typically rough and inhomogeneous, which significantly complicates dissolution, adsorption, leaching and other physio-chemical processes related to geological phenomena.

The development of the microfluidic device of the present invention has come out of efforts to determine the benefits of using microfluidics in mineral leaching processes for investigating reaction pathways and mechanisms, for correlative surface analysis, and for the high throughput screening of both chemical and physical parameters that influence the leaching rate and process relevant to, for example, AMD and its environmental concerns. In this respect, it must be appreciated though that the scope of protection afforded to the microfluidic device of the present invention is not to be limited by this development background. The microfluidic device of the present invention may find use in relation to the analysis of any particulate sample, not just a sample of a mineral ore body, and not just for the purposes of AMD assessment.

Before turning to a summary of the present invention, it is also to be appreciated that various directional terms, such as upper, lower, upwardly, upright, bottom and the like, have been used throughout this specification to provide context and clarity for the invention with reference to the normal upright use of a microfluidic device, typically on a flat surface. These terms are not to be taken as limiting the invention to be used only in one particular orientation.

The discussion of the background to the invention herein is included to explain the context of the invention. This is not to be taken as an admission that any of the material referred to was published, known or part of the common general knowledge as at the priority date of this application.

### SUMMARY OF THE INVENTION

The present invention provides a microfluidic device for analysis of a particulate sample, the device including at least one upper sample chamber with a reagent inlet and a sealable upper opening for loading sample in the sample chamber, and at least one lower flow chamber with an analyte outlet, wherein:
a) the sample chamber includes a liquid pervious floor upon which, in use, the sample will rest; and
b) the flow chamber includes spaced upright members therein, the upright members having upper surfaces, at least a portion of the upper surfaces together forming the liquid pervious floor of the sample chamber, with the spaces between the upright members forming microchannels in fluid communication with the analyte outlet.

The present invention also provides a method of analysing a particulate sample using a microfluidic device, the method including the steps of:
a) loading a particulate sample into a sealable upper opening of an upper sample chamber of the device, to rest upon a liquid pervious floor of the sample chamber;
b) passing reagent through a reagent inlet in the sample chamber to flow through the device and react with the sample to form an analyte;
c) passing analyte and unreacted reagent (if any) through the liquid pervious floor into a lower flow chamber of the device, the flow chamber including spaced upright members therein, the upright members having upper surfaces that together form the liquid pervious floor of the sample chamber, with the spaces between the upright members forming microchannels in fluid communication with an analyte outlet in the flow chamber; and
d) passing analyte and unreacted reagent through the microchannels and out the analyte outlet for subsequent analysis.

The microfluidic device and method of the present invention can be used in a wide variety of applications. Use may occur in the laboratory or in the field, but more preferably will occur in the field due to their unique features, particularly for fast testing with minimal reagent and minimal particulate sample. It is envisaged that a major use will be in the mining industry where fast, easy and remote testing of rock samples, whether they are straight from the ground during exploration or waste testing, or whether they are already beneficiated and/or processed to at least some extent, is highly desired. It is thus envisaged that one of the major reactions that would be occurring in the sample chamber between the particulate sample and the reagent will be leaching.

Preferred mineral leaching applications include reaction kinetics monitoring, leaching conditions screening and leaching mechanism studies. An example of such an application is to employ the microfluidic device for reaction conditions screening for the prediction of acid mine drainage formation during mineral processing operations. Another leaching example (a non-mineral example) is that the microfluidic device could be used for studying the controlled delivery of a drug from pellets, where it is desired to control the exact amount, rate, and/or time of delivery of the drug.

In this respect, the particulate sample may be a sample with pharmaceutical properties, a reagent that simulates a biological environment reagent may be used, and subsequent analysis may be of the pharmaceutical release, including dissolution and release kinetics monitoring and mechanism studies.

Further, the particulate sample may be a soil sample containing agricultural chemicals, soil contaminant, or naturally present chemical, the reagent may simulate environmental events, such as rain, flooding, or irrigation, and the subsequent analysis may be of the dissolution or release of the dissolved soil component, including dissolution and release kinetics monitoring and mechanism studies. The particulate sample may also be catalysts or absorbents, or any other solid-liquid interface reaction system. The particulate sample may also be more than one substance, either simply mixed together or suitably layered.

The reference in this specification to a “particulate” sample is intended to distinguish the microfluidic device of the present invention, and its use, from microfluidic devices that are for use solely with liquid samples. It is expected that the microfluidic device of the present invention will most often be used with solid particulate samples, such as ore samples for the mining industry or pelleted drugs for use in the pharmaceutical industry, however other particulate samples that are not typically regarded as being “solid” may also find use with the device of the present invention. For example, gel-filled rigid particles might be used by the pharmaceutical industry, which might also benefit from the types of analysis made possible by use of the device of the present invention.

With this in mind, it is submitted that the presence in the sample chamber of the fluid pervious floor upon which, in use, the particulate sample will rest, and the synergism between the pervious floor and the particulate sample, will itself provide a suitable definition for types and sizes of particulate samples suitable for use with the present invention. The aim is to retain generally all of the particulate sample in the sample chamber, preventing passage of particles through the pervious floor and into the flow chamber, by the use of suitably sized fluid openings in the pervious floor relative to the size of the particles in the particulate sample. The pervious floor thus functions as a type of filter with respect to the liquid passing into the flow chamber, in terms of avoiding the presence of particles in that liquid. Particles can obstruct subsequent optical analysis of the analyte and can and foul downstream electrodes that might be present, for example. Also, it will normally be undesirable to lose material from the sample chamber (and from the original particulate sample), as it will usually be necessary to know how much material was present, for example, during leaching.

The type and size of sample that is retained in this way will thus be the type and size of particulate sample that the microfluidic device and method of the present invention relates to.

The upright members of the flow chamber of the microfluidic device of the present invention will preferably be an array of micropillars, being individual columnar members with either a circular, square, rectangular, oval or other suitable cross-section, whereby the spaces between the micropillars form a regular series of microchannels therebetween. Alternatively, the upright members may be a random or ordered series of micro-walls or micro-ridges, between which suitable microchannels are formed that permit a continuous flow of liquid therethrough. In either alternative, the upright members will have an upper surface, which is ideally a generally flat surface that is able to form, together with other adjacent upper surfaces, and in conjunction with the spaces between the upright members, the abovementioned liquid pervious floor capable of supporting the particulate sample in the sample chamber.

In a first, but not necessarily a main, form of the invention, the microfluidic device will include one upper sample chamber and one flow chamber, at least a portion of the upper surfaces of the upright members in the single flow chamber forming the liquid pervious floor of the single sample chamber. In this form, the area of the sample chamber will ideally be the same or less than the area of the flow chamber, with the area of the flow chamber preferably being from about 40 mm² to about 100 mm². In this form, the area of the single sample chamber may be between about 5 to 95% of the area of the flow chamber, although it is envisaged to likely be between about 5 to 20% of the area of the flow chamber.

In a second form of the invention, the microfluidic device will include multiple upper sample chambers, each with a liquid pervious floor and a reagent inlet, and a single flow chamber, at least a portion of the upper surfaces of the upright members in the flow chamber forming the liquid pervious floors of the sample chambers. In this form, the total area of all sample chambers is preferably less than the area of the flow chamber, with the area of the flow chamber preferably being from about 40 mm² to about 100 mm² or more.

In a third form of the invention, the microfluidic device will include multiple upper sample chambers, each with a liquid pervious floor and a reagent inlet, and multiple flow chambers, each with upright members and microchannels, one sample chamber being in fluid communication with one flow chamber, and the flow chambers being in fluid communication with the analyte outlet either in series or in parallel. In this form, the area of one sample chamber will preferably be the same or less as the area of the flow chamber that it is in fluid communication with.

In all three of these alternative forms of the microfluidic device, the total volume of the device's sample chambers will preferably be from about 50 microlitres to about 800 microlitres.

In relation to the upright members of the microfluidic device of the present invention, when the upright members are micropillars, micro-walls or micro-ridges, the height of the micropillars, micro-walls and micro-ridges is preferably between about 1 and 100 micrometres, or alternatively between about 10 and 50 micrometres. Further, the diameter or thickness of the micropillars/micro-walls/micro-ridges is preferably between about 1 and 100 micrometres, or alternatively between about 6 and 30 micrometres. Further still, the spacing between the micropillars/micro-walls/micro-ridges is preferably between about 1 and 100 micrometres, or alternatively between about 6 and 30 micrometres. However, having described these preferred dimensions, reference is again made to the above comments regarding the particle size in the particulate sample and its relationship to the spacing and microchannel sizes in the flow chamber, and the opening sizes in the liquid pervious floor.

In a preferred form, the microfluidic device is a multi-layer device that includes an upper layer that provides the sample chamber and a base layer that provides the flow chamber. The base layer thus includes the upright members of the flow chamber and acts as a support for the upper layer, with the interface between the upper layer and the base layer being the plane in which the upper surfaces of the upright members of the flow chamber lie, thus defining at that interface the liquid pervious floor of the sample chamber.

Any material which can serve as a support for the upper layer may be used to form the base layer, ideally one that is suitable for etching, machining or modelling, and that is impermeable to the particulate samples being analysed and the reagents being used. Examples of suitable materials include all types of glasses, ceramics, metals and polymers. It will be understood that the selection of an appropriate material will depend upon the application. Glass materials will preferably be adopted where optical observation is required during the operation of the method.

In relation to the reagent inlet and analyte outlet of the microfluidic device, in a preferred form the reagent inlet will pass through either a side wall of the upper layer or the top wall of the upper layer, but will ideally be positioned near the sealable upper opening (and ideally through the removable cover) so as to permit entry of reagent directly into the sample chamber. Similarly, in a preferred form, the analyte outlet will be positioned to pass from the flow chamber out through either a sidewall of the base layer or the upper layer, or through the top wall of the upper layer, two of these alternatives thus requiring the analyte outlet to pass upwardly through the upper layer. These preferred locations of the reagent inlet and the analyte outlet permit the microfluidic device to be operated in an upright orientation, flat on a surface, with inlets and outlets easily accessible from above.

In relation to the upper layer, which provides the sample chamber, suitable materials will include glass, a ceramic, a metal, or polymers such as polydimethylsiloxane (PDMS), polymethyl methacrylate (PMMA), polycarbonate, a cyclic olefin copolymer, polytetrafluoroethylene (PTFE), an epoxy-based negative photoresist resin (such as the resin known as SU-8), polyimides, hydrogels, and the like. PDMS and PMMA are typically preferred due to their easy and inexpensive fabrication and chemical compatibility.

The thickness of the upper layer can vary from approximately several hundreds of micrometres to several millimetres, depending on the microfluidic device's application. In general, changing the thickness of the upper layer or the diameter (area) of the sample chamber affects the volume of the sample chamber and thus the loading capacity of the microfluidic device. Devices for mineral leaching applications can be made in the order of a few millimetres or less in all dimensions, but under a consideration of adequate representativity of the samples. The diameters of the reagent inlet and the analyte outlet are able to be varied as necessary to be between few hundreds of micrometres to few millimetres.

The sample chamber of the upper layer is of course where any dissolution/corrosion reaction occurs during operation. A leaching reagent can be introduced into the device by pumping through capillary tubing. The analyte (upon dissolution) will be contained in the flowing liquid and collected at the analyte outlet. The size of the sample chamber, its packing density and the flow rate assist in controlling the time of release or exposure of the substances, and the reaction rate, pH, temperature, and other solution properties can be monitored during the process. In addition, the microfluidic device of the present invention is capable of screening multiple variables of the same or different nature within a single device. Also, in some applications, thermal reactions can be conducted, allowing consideration of heat resistance, again depending on the particulate sample and the materials of the device.

The microfluidic device of the present invention may also be integrated with an online detection system, for example an electrochemical/light sensor for detecting the analytes in the liquid flowing through the device, such as through windows on an optically transparent base layer to provide quantitative or qualitative data in a more efficient manner.

The microfluidic device of the present invention may thus include one or more associated detection devices and/or one or more associated analysis devices, and/or one or more associated pumping systems. In this respect, such associated devices may be based upon one or more of optical absorbance, fluorescence, transmission, Raman or emission spectroscopy (including surface-enhanced Raman), or electrochemical sensors, including redox, impedance or conductivity sensors, or the like, or upon refractive index. In a preferred form, these associated devices may be formed integrally with the microfluidic device of the present invention. Alternatively, they may be separate devices that are connected in fluid communication with the microfluidic device, either in series or in parallel, as required.

### BRIEF DESCRIPTION OF DRAWINGS

Having briefly described the general concepts involved with the present invention, a preferred embodiment will now be described that is in accordance with the present invention. However, it is to be understood that the following description of the drawings and examples is not to limit the generality of the above description.

In the drawings:

FIG. 1 is a schematic illustration of the workflow for a preferred embodiment of microfluidic device in accordance with a preferred embodiment of the present invention, showing a single sample chamber and upright members in the form of micropillars (inset: an image of the experimental set-up with inlet and outlet tubing).

FIGS. 1a and 1b are alternative embodiments of microfluidic devices that are also in accordance with the present invention.

FIG. 2 shows results from the experimental work with the embodiment of FIG. 1, in particular a comparison of the sulphur (A) and iron (B) release rate from pyrite as a function of time without surface treatment and with surface treatment.

FIG. 3 also shows results from the experimental work with the embodiment of FIG. 1, in particular aqueous iron and sulphur released from pyrite as a function of pH at room temperature.

FIG. 4 also shows results from the experimental work with the embodiment of FIG. 1, in particular a 3D graph of the dissolution rate of pyrite as a function of temperature and ferric ion concentration.

FIG. 5 also shows results from the experimental work with the embodiment of FIG. 1, in particular fitted sulphur (2p) XPS spectra of the pyrite samples treated at different conditions, where the colour scheme is: red, disulphide; green, sulphide; yellow, sulphate; blue, elemental sulphur and purple, polysulfide with dotted line showing the doublet peak of each sulphur species of the same colour.

### DETAILED DESCRIPTION OF PREFERRED EMBODIMENTS

For a preferred embodiment of a microfluidic device in accordance with the present invention, a robust microfluidic device and method were developed for screening geological phenomena that occurs at the solid/liquid interface of rock ore samples as received. Evaluation of mineral dissolution/leaching for a range of reaction conditions were carried out using real rock samples with species diversity. For the purposes of illustration, screening was for acid mine drainage (AMD) under typical environmental conditions in the field. However, it will be appreciated that the inventive microfluidic device and method can also be applied to the optimization of industrial leach processes at mineral processing plants, and indeed also to non-mineral situations.

Pyrite (FeS₂) is the most abundant sulphide mineral in the earth's crust and is a primary contributor to AMD and the consequent metal release of sulphide bearing mining wastes. The rate of pyrite oxidation and the resulting acid production is dependent on various environmental factors that are dynamic and often vary substantially between regions. Here, ferric ion concentration, pH, and temperature (which are the common factors affecting the oxidation of pyrite) were examined by loading a single sample chamber in a microfluidic device with a particulate sample in accordance with this embodiment of the present invention.

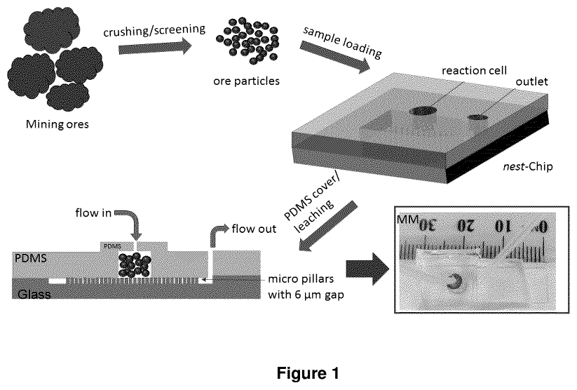

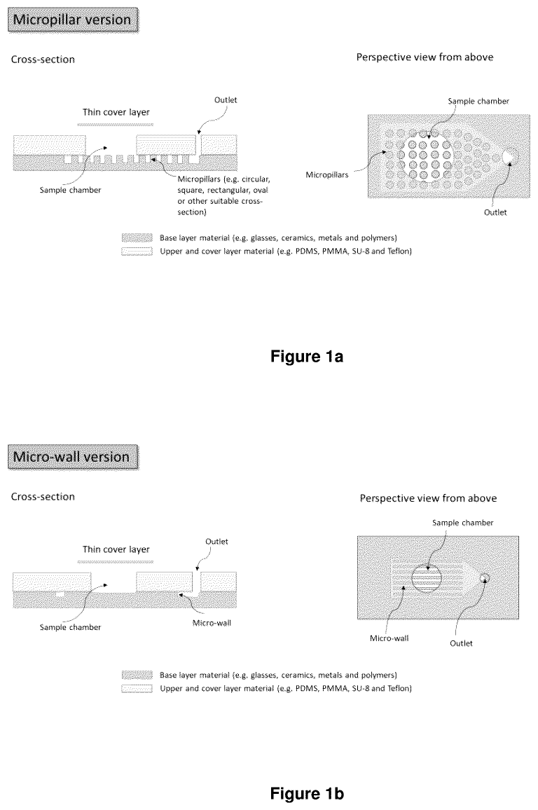

A schematic of the overall experimental setup is given in FIG. 1, with examples of suitable microfluidic devices shown in FIG. 1a and FIG. 1b.

Parallel testing in the microfluidic device offered high-throughput screening capacity. For each experiment, only 50 mg of the rock sample was required. Reagent consumption was approximately 3 mL for screening up to 6 hours of reaction time. Five parallel experiments were conducted, although the inventive method allows for greater parallelization as required. Surface characterization of the sample residue was carried out by X-ray photoelectron spectroscopy (XPS) to correlate the surface chemistry of the reaction residue with the leaching behaviour observed by solution analysis.

### Materials and Methods

Pyrite ore sample (FeS₂) was supplied by Geo Discoveries (NSW, Australia). The phase purity of the pyrite ore was confirmed by quantitative X-ray diffraction and chemical analyses. The ore was then crushed, ground and screened to a particle size range of 38-75 μm with particle surface area measured at 0.35 m²·g⁻¹. In this respect, it is envisaged that a suitable size range for most particulate samples will be from about 2 μm to about 1 mm, with a usual range likely to be from about 20 μm to about 600 μm.

Acid washing is often used for leaching experiments to remove any surface oxidised layer of ore samples that might be formed during sample preparation. However, the literature is typically silent on detail for tracking any change of leachate chemistry under these different conditions, principally due to it being difficult to access using conventional methods. It will be seen from the below description and discussion that the use of the device and method of the present invention can be advantageous for tracking the evolution of leachate chemistry under different conditions and does show that such an acid wash in fact tends to have no impact on the leach behaviour of the intrinsic mineral of examined samples.

In this respect, each pyrite ore sample was washed with 3 M HCl solution for several minutes to remove oxidised surface layers possibly formed during sample preparation.

Following sample preparation, 1 M KOH (AR, 85%) and hydrochloric acid (37%) were used to adjust the pH of a reagent solution. FeCl₃·6H₂O (AR) was used for the preparation of ferric ion concentrations. 0.1 M KCl was used as the background electrolyte to ensure the solution ionic strengths were approximately constant throughout the experiments. All chemicals were purchased from Chem-Supply, Australia. Milli-Q water (18 MΩ·cm resistivity) was used to prepare all solutions.

In order to form the upper layer of the device, a mass ratio of 10:1 of Sylgard 184 silicone elastomer base and curing agent was mixed thoroughly and poured onto a hydrophobized silicon wafer-based container. The PDMS was cured at 60° C. for 4 h, then peeled off from the silicon master. After this, the sample chamber and reagent inlet port were formed by coring 4 mm and 1.5 mm holes respectively with a biopsy punch.

The microfluidic device of the embodiment illustrated in FIG. 1 (and also FIG. 1a) was assembled by sealing this thin upper PDMS layer (thickness ˜8 mm) on a “pillar cuvette” (the base layer) with pillars having a 6 μm gap and 10 μm height through plasma bonding. The pillar cuvette was of the type described in Holzner, G.; Kriel, F. H.; Priest, C., “Pillar Cuvettes: Capillary-Filled, Microliter Quartz Cuvettes with Microscale Path Lengths for Optical Spectroscopy.” Anal. Chem. 2015, 87 (9), 4757-4764, the full content of which is incorporated herein by reference.

During plasma bonding of the upper PDMS layer with the base layer (the pillar cuvette), care was taken to align the sample chamber and reagent inlet within the area of the pillar cuvette arrangement in the flow chamber.

After loading ore samples into the sample chamber, the opening of the sample chamber was sealed with a thin layer of PDMS (a removable cover) which allowed introduction to the sample chamber via the reagent inlet and TYGON® tubing, together with optical inspection of the sample chamber, as required.

Reagent flow through the reagent inlet and the sample chamber was driven by a peristatic pump (Gilson, Minipuls®) through capillary tubing (0.5 mm inner and 1.58 mm outer diameter) into the sample chamber at 0.65±0.05 mL/h. The leach solution (analyte) was collected at the analyte outlet through capillary tubing in a glass vial over a period of 1 h for each sample.

The analyte collected at the analyte outlet was analysed by inductively coupled plasma mass spectrometry (ICP-MS) (Agilent 8800). The average leach rate (mM·m⁻²·s⁻¹) during the collection period (1 h per measurement) was determined from the ion concentration in the collected analyte and from the sample surface area. Screening experiments were repeated three times with similar results, showing an experimental error within 6%.

After the leach, the ore samples were rinsed with milli-Q water to remove any residue leachate. The removable PDMS cover was then removed from the sample chamber and the wet samples were quickly transferred into a small plastic vial with rinsing water and stored in the freezer for cold stage (−134° C.).

XPS spectra were collected using a Kratos AXIS Ultra DLD spectrometer. The x-ray was a mono-chromatic aluminum x-ray running at 225 W with a characteristic energy of 1486.6 eV. The area of analysis (Iris aperture) was a 0.3 mm×0.7 mm slot; the analysis depth was approximately 15 nm into the surface of the sample. The analysis vacuum was 4×10⁻⁸ Torr. The electron take-off angle was normal to the sample surface. Spectra were interpreted using the software package CasaXPS.

### Results and Discussion

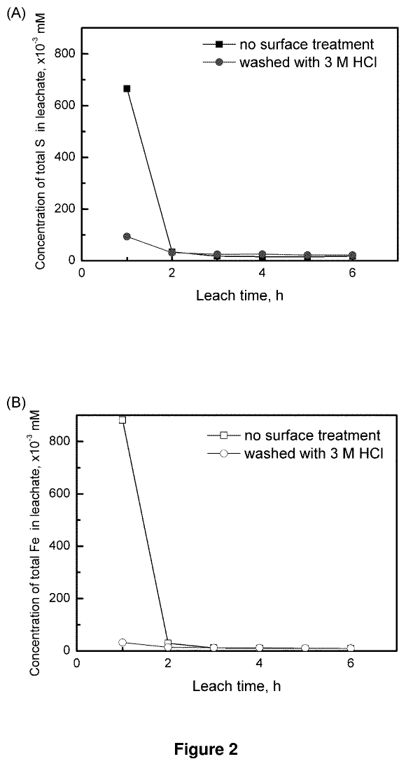

Pyrite dissolution kinetics and surface pre-treatment—The flow chemistry applied in the microfluidic device of the invention enables the examination of reaction dynamics, particularly in the early stages of leaching experiments, which are difficult to access using conventional methods. FIG. 2 shows the evolution of the leachate chemistry for both dissolved iron and sulphur in pyrite as a function of time (up to 6 hours). A rapid decline in both sulphur and iron in the leachate was observed for the untreated pyrite sample compared to the leached sample in the first two hours, suggesting that the leaching removed the more reactive surface layer that contains both iron and sulphur species. The leachate chemistry (for sulphur and iron respectively) was quite stable and very similar at the later stage of leaching for both untreated and treated samples. This may be attributed to the dissolution of fresh pyrite exposed during the course of leaching. The Fe/S ratio (0.41-0.46) observed at a later stage in the leachate for both pyritic samples is slightly below the stoichiometric dissolution of pyrite (0.5).

Generally, the result shows that that an acid wash (as a pre-treatment) has no impact on the leach behaviour of intrinsic pyrite mineral. Therefore, for further investigation of AMD formation under various conditions, an acid wash (a pre-treatment) was used as standard sample treatment before running experiments. Further details about the surface chemistry of the outer layer of pyrite before and after leaching will be discussed below. No surface passivation was observed under the conditions examined during the leach time.

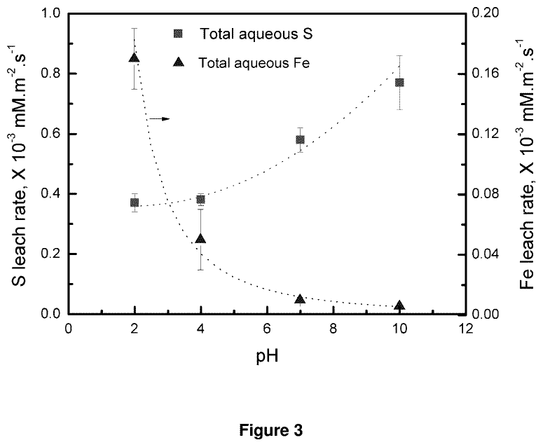

Effect of pH—the effect of pH on the dissolution rate of pyrite was examined in the range of pH 2-10. The leach rates of each element at different pH were calculated from the measured solution total sulphur or iron concentration divided by the sample's total surface area (determined from the mass and specific surface area of the sample) and the collection time for each measurement (1 h). The average value of the leach rate at steady state (after initial removal of the oxidation layer) was then plotted against pH (error bars represent the standard deviation of the obtained leach rates), as shown in FIG. 3.

FIG. 3 shows that the pH has a significant but very different impact on the leach (release) rate of sulphur and iron from pyrite. The higher the pH, the higher the concentration of sulphur compounds in solution. However, the concentration of iron detected in solution was decreased as the pH increased. When the pH was higher than 7, only trace amounts of iron were detected in solution. The Fe/S ratio calculated from the total iron and sulphur species detected in the solutions declined from 0.46 to 0.008 as the pH increased from 2 to 10, i.e. the pyrite surface did not exhibit the expected stoichiometric dissolution (Fe/S=0.5) at high pH. This is most probably due to the formation of iron precipitate as iron (hydro)oxides on the pyrite surface.

As the pH went higher, more iron (hydro)oxides grew at the pyrite surface. However, this did not prevent further pyrite oxidation because sulphur species were still released to the solution at an increasing rate, as shown in FIG. 3. The reaction rate obtained is around four orders of magnitude higher than expected, probably due to the continuous flow of fresh leaching solution and the continuous removal of by-products from the pyrite sample.

The log rate of pyrite dissolution (mol·m⁻²·s⁻¹) plotted against pH showed a reaction order for pH of 0.04 (with a R square factor of 0.96) in the range of pH 2-10, which vary widely between 0.11 to 0.5. The small reaction order of the present study for H+ indicates that pH has a lesser effect on the observed rate compared to bath-scale experiments, due to the continuous removal of iron precipitates (formed at higher pH) in the microfluidic flow system avoiding possible surface passivation. The sulphur concentration detected in the leachate was applied for the calculation of pyrite dissolution rate instead of iron due to the precipitation of iron species at the pyrite surface during leaching at higher pHs.

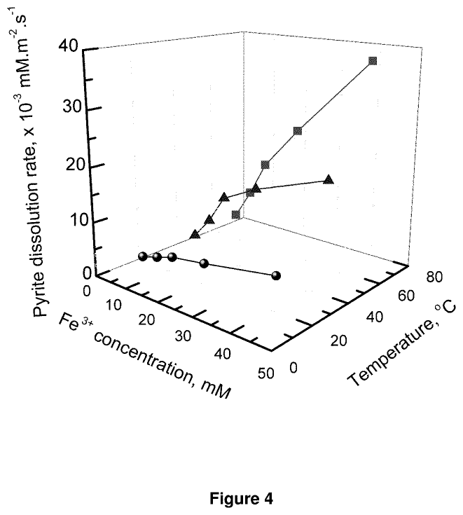

Combinatorial screening (temperature, ferric ion concentration and time)—benefiting from the minimization of the reaction system, the microfluidic device of the invention is attractive for the screening of multiple variables of the same or a different nature within a single device. In this study, ferric ion concentration and temperature were chosen as screening parameters for the study of acid mine drainage (AMD). With this in mind, each sample chamber was exposed to different leach conditions, enabling rapid parameter screening (results shown in FIG. 4).

FIG. 4 shows the screening results for varied ferric ion concentration (0, 5, 10, 20, and 40 mM ferric ion concentration in the fresh leach solution) for three different temperatures (23, 50 and 75° C.). The average pyrite dissolution rates were determined at steady state (observed between 3 to 6 h) based on the sulphur concentration measured in the leachate. The relationship between the leach rate and the two variables (temperature and Fe³⁺ concentration) is clearly seen in FIG. 4.

Increasing either variable increases the leach rate, but using a fraction of the sample, reagent and time. Surface passivation was not observed under the examined conditions, which is likely due to the continuous flow of fresh leach solution. On the basis of the above results, the reaction order of pyrite dissolution rate on Fe³⁺ concentration was calculated as 0.72±0.06, which was as expected. The Arrhenius plot shows the apparent activation energy of the pyrite oxidation reaction by ferric ion solution is around 30.6±0.7 kJ·mol⁻¹, again being consistent with expected values ranging between 33 to 63 kJ·mol⁻¹.

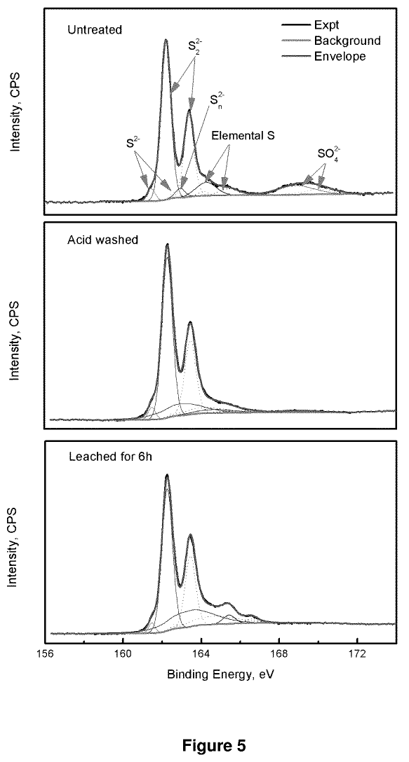

Surface analysis by XPS—due to the formation of iron precipitate on the pyrite surface during leaching, analysis of the sulphur signal is practically more important than that of iron. FIG. 5 shows sulphur (2p) spectra of pyrite samples treated at different conditions: untreated, acid washed (3 M HCl) and leached for 6 h at pH 2. For all pyrite samples, the characteristic peak located at 162 eV in the sulphur (2p) spectra agrees with the expected value for pyritic S₂²⁻. A small S⁻² peak at 161 eV was also found on all samples. Signals detected in the range of 164 to 165 eV suggested the presence of elemental sulphur and Sₙ²⁻ at the surface of all pyrite samples. On untreated samples, there was a peak at 168 eV, attributed to the presence of sulphate species on the surface. This peak intensity was significantly decreased after the acid wash, suggesting sulphate species were removed from the surface during washing. After leaching for 6 h, the peak at 168 eV was barely observed, indicating a complete dissolution of sulphates from the surface. No traces of sulphites were found in the 166 to 167 eV range. These results are in good agreement with the analysis of the leachate solutions collected.

### Implications

Microfluidic screening of geological phenomena such as leaching offers a rapid approach to investigating natural processes that are environmentally or commercially important. The complex parameter space encountered in these reaction systems demands high throughput multiparameter screening, using minimal sample, reagent and time. The microfluidic approach able to be used by the adoption of the microfluidic device and method of the present invention meets these demands and is shown to report meaningful, time-resolved results for various reaction conditions.

Mineral samples in particulate form can be directly loaded into the device, without the need for flat, large areas of sample (e.g. polished or embedded in resin). Samples can thus be obtained direct from mine sites and, in many cases, on site screening will be possible. Low-cost testing could precede field trials, which are typically expensive and time-consuming. In addition, the method of the present invention could be used to study a wide range of solid-liquid interactions in flow (adsorption, dissolution, and other surface chemistry phenomena) across many different fields of application, including outside of the AMD and mineral processing work illustrated in these examples.

In conclusion, it must be appreciated that there may be other variations and modifications to the configurations described herein which are also within the scope of the present invention.

1. A microfluidic device for analysis of a particulate sample, the device including at least one upper sample chamber with a reagent inlet and a sealable upper opening for loading sample in the sample chamber, and at least one lower flow chamber with an analyte outlet, wherein:
a) the sample chamber includes a fluid pervious floor upon which, in use, the sample will rest; and
b) the flow chamber includes spaced upright members therein, the upright members having upper surfaces, at least a portion of the upright surfaces together forming the fluid pervious floor of the sample chamber, with the spaces between the upright members forming microchannels in fluid communication with the analyte outlet.

2. A device according to claim 1, wherein the upright members are an array of micropillars, in the form of individual columnar members with either a circular, square, rectangular, oval or other suitable cross-section, whereby the spaces between the micropillars form a regular series of microchannels therebetween.

3. A device according to claim 1, wherein the upright members are a random or ordered series of micro-walls or micro-ridges, between which suitable microchannels are formed that permit a continuous flow of liquid therethrough.

4. A microfluidic device according to claim 1, including one upper sample chamber and one flow chamber, at least a portion of the upper surfaces of the upright members in the flow chamber forming the fluid pervious floor of the sample chamber.

5. A microfluidic device according to claim 4, wherein the area of the sample chamber is the same or less than the area of the flow chamber.

6. A microfluidic device according to claim 4, wherein the area of the flow chamber is from about 40 mm² to about 100 mm².

7. A microfluidic device according to claim 4, wherein the volume of the sample chamber is from about 50 microlitres to about 800 microlitres.

8. A microfluidic device according to claim 1, including multiple upper sample chambers, each with a fluid pervious floor and a reagent inlet, and a single flow chamber, at least a portion of the upper surfaces of the upright members in the flow chamber forming the fluid pervious floors of the sample chambers.

9. A microfluidic device according to claim 8, wherein the total area of all sample chambers is less than the area of the flow chamber.

10. A microfluidic device according to claim 8, wherein the area of the flow chamber is from about 40 mm² to about 100 mm².

11. A microfluidic device according to claim 8, wherein the total volume of all sample chambers is from about 50 microlitres to about 800 microlitres.

12. A microfluidic device according to claim 1, including multiple upper sample chambers, each with a fluid pervious floor and a reagent inlet, and multiple flow chambers, each with upright members and microchannels, one sample chamber being in fluid communication with one flow chamber, the flow chambers being in fluid communication with the analyte outlet either in series or in parallel.

13. A microfluidic device according to claim 12, wherein the area of one sample chamber is the same as the area of the flow chamber that it is in fluid communication with.

14. A microfluidic device according to claim 12, wherein the total area of all flow chambers is from about 40 mm² to about 100 mm².

15. A microfluidic device according to claim 12, wherein the total volume of all sample chambers is from about 50 microlitres to about 800 microlitres.

16. A microfluidic device according to claim 1, wherein the upright members are micropillars and the height of the micropillars is between about 1 and 100 micrometres, the size of the micropillars is between about 1 and 100 micrometres, and/or the spacing between the micropillars is between about 1 and 100 micrometres.

17. A microfluidic device according to claim 1, wherein the sealable upper opening of the or each sample chamber is removable.

18. A microfluidic device according to claim 1, including one or more integrated detection devices and/or one or more integrated analysis device.

19. A microfluidic device according to claim 18, wherein the integrated detection devices and integrated analysis devices include optical absorbance, fluorescence, transmission, Raman or emission spectroscopy, or electrochemical sensors, including redox, impedance or conductivity sensors, or the like, or upon refractive index.

20. (canceled)

21. A method of analysing a particulate sample using a microfluidic device, the method including the steps of:
a) loading a particulate sample into a sealable upper opening of an upper sample chamber of the device, to rest upon a fluid pervious floor of the sample chamber;
b) passing reagent through a reagent inlet in the sample chamber to flow through the device and react with the sample to form an analyte;
c) passing analyte and unreacted reagent through the fluid pervious floor into a lower flow chamber of the device, the flow chamber including spaced upright members therein, the upright members having upper surfaces that together form the fluid pervious floor of the sample chamber, with the spaces between the upright members forming microchannels in fluid communication with an analyte outlet in the flow chamber; and
d) passing analyte and unreacted reagent through the microchannels and out the analyte outlet for subsequent analysis.

22. A method according to claim 20, wherein the particulate sample is a mineral ore, soil, a chemical, biological material, or a pharmaceutical.

23. A method according to claim 20, wherein the particulate sample is a rock sample from a mineral ore body, the reagent is a leaching reagent, and the subsequent analysis is of the leaching of the ore body, including reaction kinetics monitoring, leaching conditions screening and/or leaching mechanism studies.

24. A method according to claim 23 wherein the particulate sample is a sulphide-bearing mining waste derived from the processing of a pyrite mineral, and the subsequent analysis is reaction conditions screening to predict acid mine drainage formation as an outcome of mineral processing.

25. A method according to claim 20, wherein the particulate sample is a sample with pharmaceutical properties, the reagent simulates a biological environment reagent, and the subsequent analysis is of the pharmaceutical release, including dissolution and release kinetics monitoring and mechanism studies.

26. A method according to claim 20, wherein the particulate sample is a soil sample containing agricultural chemicals, soil contaminant, or naturally present chemical, the reagent simulates environmental events, such as rain, flooding, or irrigation, and the subsequent analysis is of the dissolution or release of the dissolved soil component, including dissolution and release kinetics monitoring and mechanism studies.
